(19) United States
(12) Patent Application Publication
(10) Pub. No.: US 2002/0095110 A1
(43) Pub. Date: Jul. 18, 2002

(54) Stabilized transmyocardial implant

(76) Inventors: Guy P. Vanney, Blaine, MN (US); Robert A. Eno, Plymouth, MN (US); Dale M. Groth, St. Louis Park, MN (US)

Correspondence Address: MERCHANT & GOULD PC, P.O. BOX 2903, MINNEAPOLIS, MN, 55402-0903, US

(21) Appl. No.: 09/975,746

(22) Filed: Oct. 10, 2001

Related U.S. Application Data
(60) Provisional application No. 60/304,165, filed on Oct. 11, 2000.

(51) Int. Cl. A61F 2/06

(52) U.S. Cl. 604/8; 623/1.36

(57) ABSTRACT

A transmyocardial implant for establishing a blood flow path through a myocardium between a heart chamber and a lumen of a coronary vessel residing at an exterior of said wall includes a hollow conduit having a vessel portion and a myocardial portion. The vessel portion is sized to be received within the lumen. The said myocardial portion is sized to extend from the vessel through the myocardium and into the chamber. The conduit has an open first end and an open second end on respective ones of the vessel and myocardial portions to define a blood flow pathway within an interior of the conduit between the first and second end. At least the myocardial portion of the conduit is formed of a conduit material sufficiently rigid to resist deformation and closure of the pathway in response to contraction of the myocardium. The conduit material is resistant to thrombus formation. An anchor is secured to the conduit and positioned to overlie and be secured to an epicardial surface of the myocardium when the vessel portion of the conduit is secured within the lumen.

### CROSS-REFERENCE TO RELATED APPLICATIONS

This application claims priority to U.S. application Ser. No. 09/686,251 filed Oct. 11, 2000 and later converted to U.S. Provisional Application Serial No. 60/304,165.

### BACKGROUND OF THE INVENTION

1. Field of the Invention

This invention pertains to an implant for directing blood flow directly between a chamber of the heart and a coronary vasculature. More particularly, this invention pertains to such an implant with an enhanced design for securing placement of the implant.

2. Description of the Prior Art

Commonly assigned U.S. Pat. Nos. 5,755,682 and 5,944,019 teach an implant for defining a blood flow conduit directly from a chamber of the heart to a lumen of a coronary vessel. An embodiment disclosed in the aforementioned application teaches an L-shaped implant in the form of a rigid conduit. The conduit has one leg sized to be received within a lumen of a coronary artery and a second leg sized to pass through the myocardium and extend into the left ventricle of the heart. As disclosed in the above-referenced patent application, the conduit is rigid and remains open for blood flow to pass through the conduit during both systole and diastole. The conduit penetrates into the left ventricle in order to prevent tissue growth and occlusions over an opening of the conduit.

Commonly assigned U.S. Pat. No. 5,984,956 teaches an implant such as that shown in the aforementioned '682 and '019 patents with an enhanced fixation structure. One embodiment of the enhanced fixation structure includes a fabric cuff surrounding the conduit to facilitate tissue growth on the exterior of the implant. The fabric is described as a polyester cuff having interstitial spaces into which tissue may grow.

Implants such as those shown in the aforementioned patents include a portion to be placed within a coronary vessel and a portion to be placed within the myocardium. When placing a portion of the implant in the coronary artery or other coronary vessel, the artery is incised by an amount sufficient to insert the implant. Preferably, the artery is ligated distal to an obstruction. A transverse incision is made through the artery distal to the ligation. Tools and procedures for such an implantation are shown and described in commonly assigned and copending U.S. Pat. No. 6,029,672.

In the foregoing references, a constantly open blood flow path is preferred. However, the references also teach a conduit with a valve which closes during diastole. The afore-mentioned U.S. Pat. No. 5,944,019 teaches a conduit with a valve which only partially closes during diastole to permit a washing back-flow.

Conduits which include a valve or which otherwise close during the heart cycle are shown in U.S. Pat. No. 5,287,861; U.S. Pat. Nos. 5,409,019 and 5,429,144 (all to Wilk) and PCT International Publication Nos. WO 98/08456 and WO 98/46115. The alleged benefits of a valve in such a conduit are described in Kashem et al., “Feasibility Study of Left Ventricle to Coronary Artery Perfusion for Severe Coronary Artery Diseases”, ASAIO Journal, Vol. 45, No. 2 (March-April, 1999) (Abstract).

After an implant with a fabric cuff is placed in the myocardium, the tissue of the myocardium grows into the cuff. A healing process takes place over time and includes fibrosis at the cuff. Such healing may include contraction of tissue around the implant. Such healing can cause the implant to migrate over time and be drawn in a direction into the heart chamber. Such movement can cause the implant to be moved out of axial alignment with the coronary vessel leading to occlusion of the implant.

The present invention is directed to an implant which resists forces tending to draw the implant into the heart chamber.

### SUMMARY OF THE INVENTION

According to a preferred embodiment of the present invention, a transmyocardial implant is disclosed for establishing a blood flow path through a myocardium wall between a heart chamber and a lumen of a coronary vessel residing at an exterior of said wall. The implant includes a hollow conduit having a vessel portion and a myocardial portion. The vessel portion is sized to be received within the lumen. The myocardial portion is sized to extend from the vessel through the myocardium and into the chamber. The conduit has an open first end and an open second end on respective ones of the vessel and myocardial portions to define a blood flow pathway within an interior of the conduit between the first and second end. At least the myocardial portion of the conduit is formed of a conduit material sufficiently rigid to resist deformation and closure of the pathway in response to contraction of the myocardium. An anchor is secured to the conduit and positioned to overlie and be secured to an epicardial surface of the myocardium when the vessel portion of the conduit is secured within the lumen.

### BRIEF DESCRIPTION OF THE DRAWINGS

FIG. 1 is a side elevation, schematic view of an implant without cuff in a heart wall and illustrating tilting following implant;

FIG. 2 is a side elevation view of an implant with an epicardial cuff;

FIG. 3 is a top plan view of an implant of FIG. 2 with a first embodiment of an anchor;

FIG. 4 is a side elevation view of the implant and anchor of FIG. 3;

FIG. 5 is a distal end elevation view of the implant and anchor of FIG. 3;

FIG. 6 is a top plan view of an implant of FIG. 2 with a second embodiment of an anchor;

FIG. 7 is a side elevation view of the implant and anchor of FIG. 6;

FIG. 8 is a distal end elevation view of the implant and anchor of FIG. 6;

FIG. 9 is a top plan view of an implant of FIG. 2 with a third embodiment of an anchor;

FIG. 10 is a side elevation view of the implant and anchor of FIG. 9;

FIG. 11 is a distal end elevation view of the implant and anchor of FIG. 9; and

FIG. 12 is a side elevation, cross-sectional view of an implant of FIG. 2 with a fourth embodiment of an anchor.

### DESCRIPTION OF THE PREFERRED EMBODIMENT

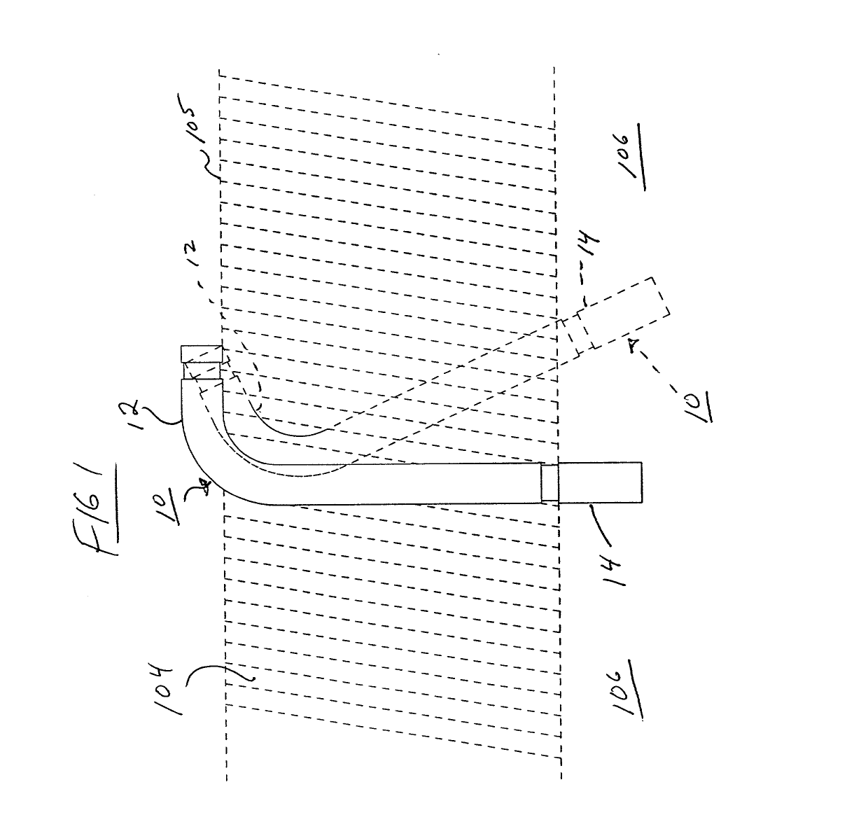

With initial reference to FIG. 1, an implant such as that described in U.S. Pat. Nos. 5,755,682; 5,944,019 and 5,984,956 (all incorporated herein by reference) is shown. In at least one embodiment, the implant includes a conduit in the form of an L-shaped tube. Such a conduit 10 is shown in FIG. 1.

The material of the conduit 10 is preferably a rigid material in order to withstand contraction forces of the myocardium. By way of non-limiting example, where the conduit is to be placed in a coronary vessel having an internal diameter not less than 2.5 mm, the tube will have an outside diameter of about 2.5 millimeters and an internal diameter of about 1.5 millimeters to provide a wall thickness of about 0.5 millimeters.

The conduit 10 has a vessel portion 12 sized to be received within the lumen of a coronary vessel such as the lumen of a coronary artery (not shown in FIG. 1 but shown in U.S. Pat. No. 5,984,956) at an upper or epicardial surface 105. The conduit 10 has a myocardial portion 14 extending at a right angle to the axis of vessel portion 12. The myocardial portion 14 is sized to extend from the coronary artery directly through the myocardium 104 and protrude into the left ventricle 106 of a patient's heart.

The tube 10 is preferably formed of titanium or other smooth biocompatible material in order to resist tissue growth on the surfaces of the conduit 10. Titanium is a presently preferred material due its long-term use in the cardiovascular industry. Further, titanium is sufficiently rigid to withstand deformation forces caused by contraction of the myocardium 104 to avoid deformation of the tube 10 so that the tube 10 remains open during both diastole and systole.

Since the titanium is resistant to thrombus formation, the titanium of the tube 10 does not fix the device within the myocardium of the patient. Therefore, as taught in U.S. Pat. No. 5,984,956, a completed implant can include a tissue growth-inducing material secured to an exterior surface of the conduit 10. Not shown in FIG. 1 but illustrated in U.S. Pat. No. 5,984,956 as a polyester fabric cuff, the tissue-growth-inducing material has interstitial spaces into which tissue of the myocardium may grow.

FIG. 1 illustrates undesirable movement of the implant 10 after surgical placement. The mechanics of the healing process and forces exerted on the device such as myocardial contraction urge the implant 10 into the myocardium 104 toward the left ventricle 106. The implant 10 responds with the myocardial portion 14 rotating distally (as shown in phantom lines in FIG. 1).

The embodiments to be described all include a short cuff 52 preferably residing only in the upper one-half to one-third thickness of the myocardium near the epicardial surface. FIG. 2 illustrates such a cuff 52. Preferably the cuff 52 has a length that is no greater than half a length of the myocardial portion 14. By way of example, the cuff 52 has an axial length of 4 mm (compared with a 15 mm length for cuffs sized to extend the full thickness of the myocardium). Such a shortened cuff for residence in the myocardium near the epicardial surface is conveniently referred to as an “epicardial cuff”. Preferably the remainder of the myocardial portion 14 not covered by the cuff 52 is highly polished to resist tissue attachment.

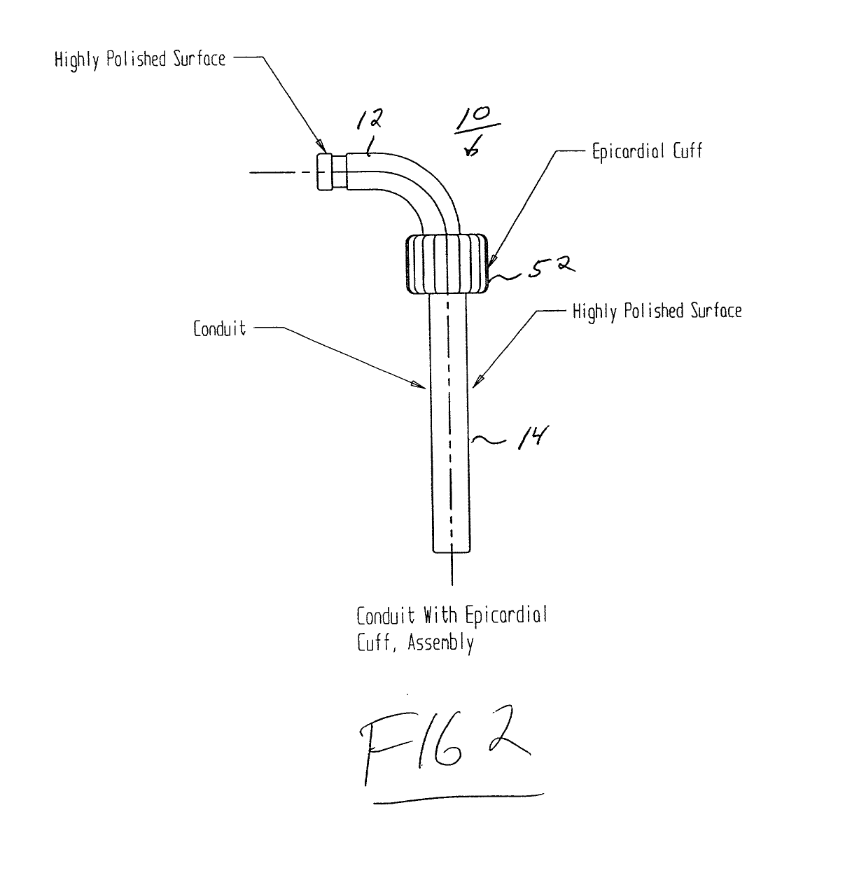

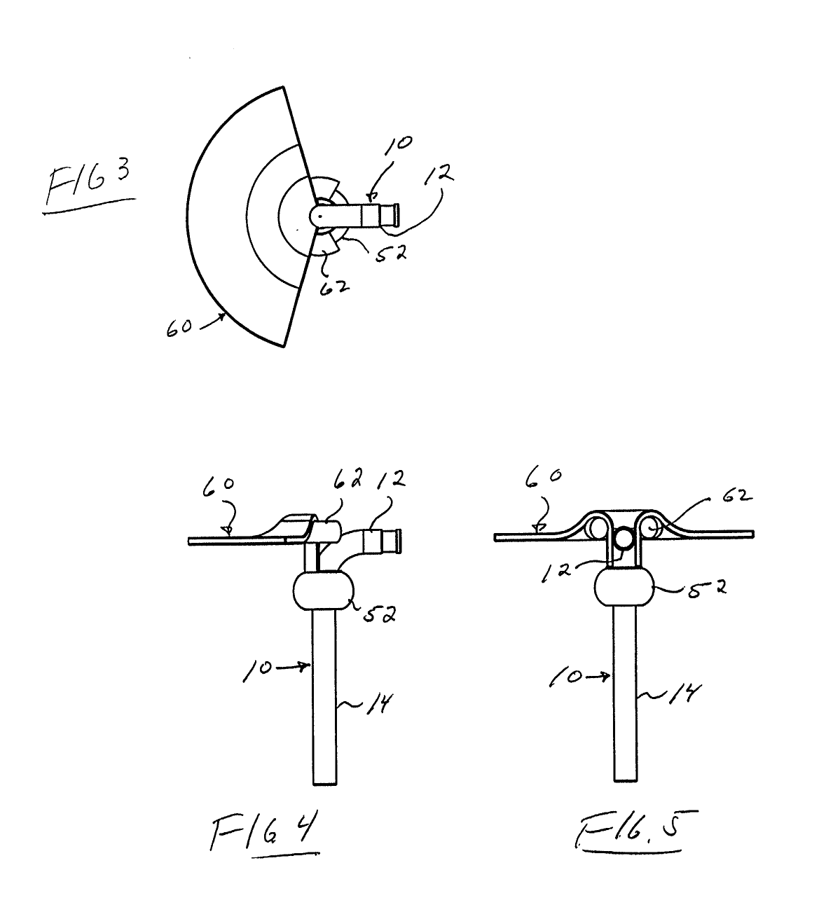

FIGS. 3-5 illustrate an embodiment where the implant 10 includes a cape 60 for resisting downward movement of the proximal side of the implant 10 and distributing the forces of such resistance over large area. In the embodiments of FIGS. 3-5 and in the remaining embodiments to be discussed, all use modifications of an implant such as that shown in FIG. 2 with an epicardial cuff 52. Accordingly, elements of such implant 10 and cuff 52 in common with such embodiments are numbered identically throughout.

In the embodiments of FIGS. 3-5, a polyester fabric cape 60 is attached to the implant 10 at the cuff 52 (e.g. by stitching the cape 60 to the cuff 52) and fans out in a plane perpendicular to the myocardial portion 14 and extending in the proximal direction of the implant 10 (i.e., in a direction opposite the distally extending vessel portion 12).

The collar 60 overlays a ring segment 62. The ring segment is PTFE or other rigid material (which may be covered with a polyester fabric). The outer perimeter of the cape 60 is sutured to the epicardial surface of the heart wall. In the event the implant 10 is subjected to forces urging the implant 10 into the myocardium, the cape 60 resists such a motion. The load of such resistance is distributed in an arch by the ring segment 62.

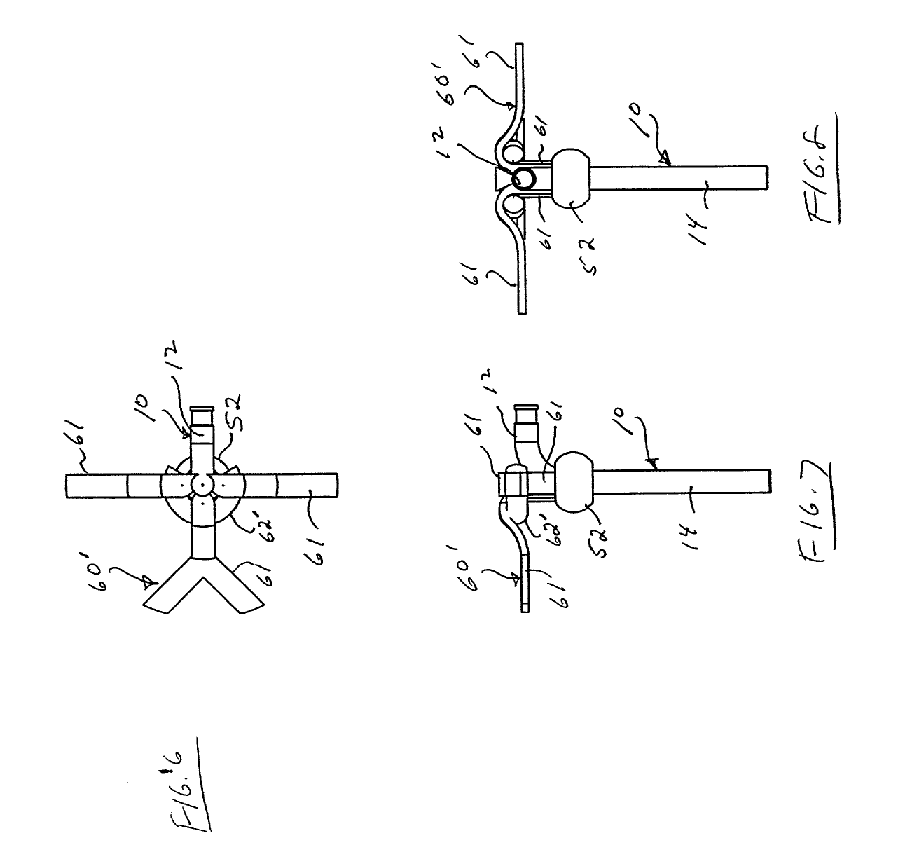

Similar to the embodiment of FIGS. 3-5, the embodiment of FIGS. 6-8 replaces the solid cape 60 with a restraint 60′ defined by a plurality of straps 61. The straps 61 extend outwardly proximally from the implant and perpendicular to the proximal-distal axis of the implant 10. The straps 61 are polyester fabric and are sutured to the epicardial surface. The straps 61 are more resistant to bunching than the cape 60 of FIGS. 3-5. The straps 61 are stitched to the collar 52 and overlie a ring 62′ similar to ring 62 of the previous embodiment. The straps radiate outwardly and to the side.

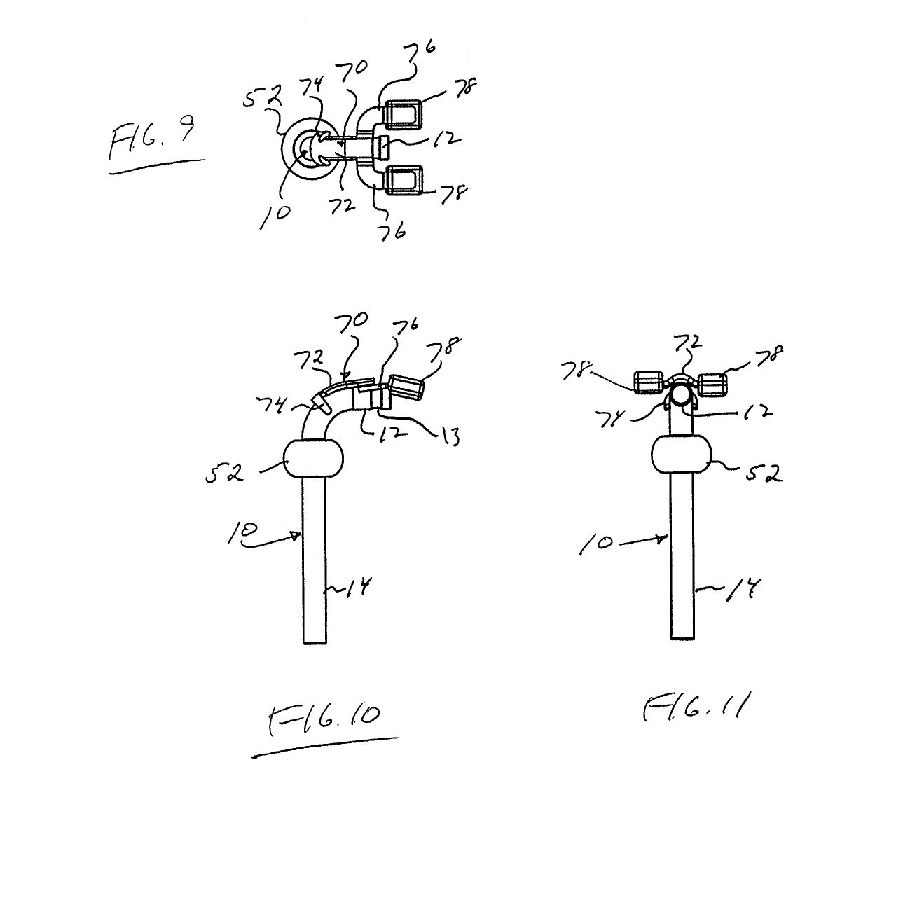

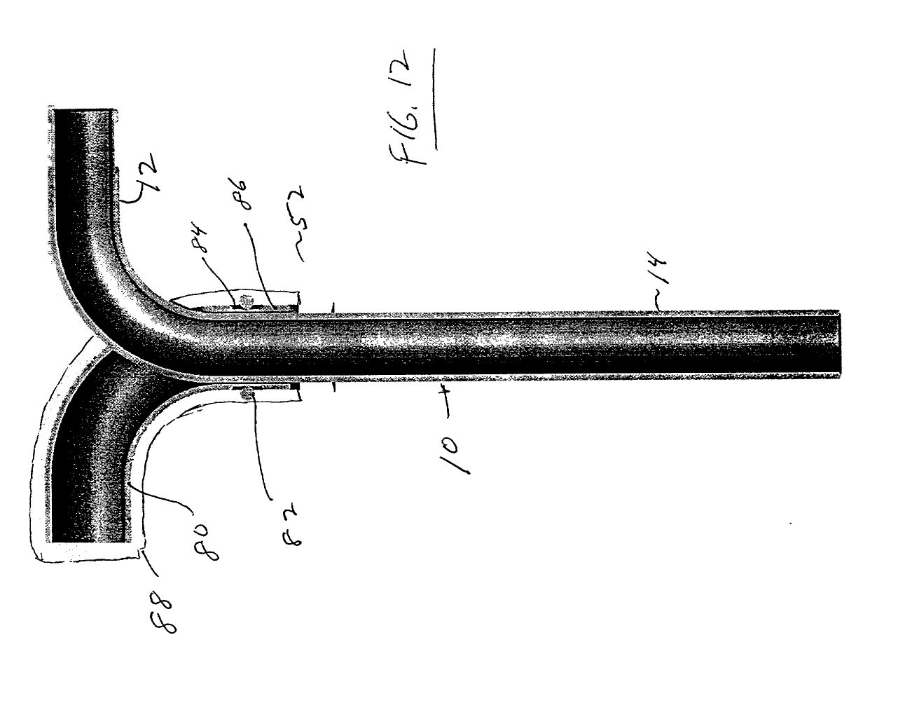

In FIGS. 9-11, an embodiment is shown with a stabilizing clip 70 attached to the vessel portion 12. The stabilizing clip 70 includes an arcuate, thin body 72 in the form of a flat plate bent to conform with the arcuate bend of the implant from the myocardial portion top the vessel portion. A snap clip 74 fastens the body 72 to the implant by being press fit onto the implant 10. Two support arms 76 are attached to the body 72. The support arms 76 are positioned to be spaced apart on opposite sides of the vessel portion 12 and extend generally parallel to an upper edge of the vessel portion. The support arms 76 extend beyond the vessel portion 12. Suture pads 78 of polyester fabric are provided on the free ends of the support arms 76. In the figures, the arms 76 are symmetrical. The arms can be asymmetrical with one longer than the other to span and clear any nearby vessels.

When using the implant 10 of the embodiments of FIGS. 9-11, the implant 10 and cuff 52 (but without attached clip 70) are placed in the heart as taught in PCT Ser. No. PCT/US99/08343. The myocardial portion 14 is placed through the myocardium and the vessel portion 12 is inserted into the coronary artery. A suture (not shown) may be placed surrounding the artery over the suture groove 13. After placement of the vessel portion 14 in the artery, the clip 70 is attached to the implant 10 by snapping the clip 74 onto the implant 10 with the pads 78 resting against the epicardial surface on opposite sides of the vessel portion 12. Sutures (not shown) may be placed surrounding the clip body 72 at snap clip 74 to prevent movement of the clip 70 on the implant 10.

The clip 70 is a brace to prevent misalignment of the vessel portion 12 and coronary vessel following implantation. The snap clip 74 permits sliding positioning of the clip 70 on the implant 10 to adjust the placement at time of surgery. This insures the surgeon may place the suture pads 78 on the epicardial surface. The surgeon sutures the pads 78 to the heart.

FIG. 12 illustrates a still further embodiment for immobilizing the implant 10. In FIG. 12, a rigid support 80 is attached to the implant 10 and extends proximally. The support 80 is shaped to approximate the shape of the vessel portion 12. A suture 82 received in a groove 84 of a flexible mounting sleeve 86 anchors the support 80 to the implant 10. A polyester sleeve 88 surrounds the support 80. A suture (not shown) will surround the sleeve 88 to attach the support to the epicardial surface of the heart. As the implant 10 is urged to tilt, the support 80 abuts the heart and resists such tilting.

Having disclosed the present invention in a preferred embodiment, it will be appreciated that modifications and equivalents may occur to one of ordinary skill in the art having the benefits of the teachings of the present invention. It is intended that such modifications shall be included within the scope of the claims which are appended hereto.

What is claimed:

1. A transmyocardial implant for establishing a blood flow path through a myocardium wall between a heart chamber and a lumen of a coronary vessel residing at an exterior of said wall, said implant comprising:
a hollow conduit having a vessel portion and a myocardial portion, said vessel adapted to be placed in fluid communication with said lumen and said myocardial portion sized to extend from said vessel through said myocardium wall into said chamber, said conduit having open first and second ends on respective ones of said vessel and myocardial portions to define a blood flow pathway within an interior of said conduit between said first and second ends;
at least said myocardial portion of said conduit formed of a conduit material sufficiently rigid to resist deformation and closure of said pathway in response to contraction of said myocardium and said conduit material resistant to thrombus formation; and
an anchor secured to said conduit and positioned to overlie and be secured to an epicardial surface of said myocardium when said vessel portion of said conduit is secured within said lumen.

2. An implant according to claim 1 further comprising a tissue growth inducing material secured to an exterior of said myocardial portion, said tissue growth inducing material covering at most only half of said myocardial portion.

3. An implant according to claim 1 wherein said tissue growth inducing material is a polyester fabric.

4. An implant according to claim 1 wherein said anchor includes an anchor material extending from said conduit opposite a direction of said vessel portion.

5. An implant according to claim 4 wherein the myocardium wall includes an exterior epicardial surface, and wherein said anchor material is a sheet of flexible material adapted to extend over a surface area of said epicardial surface opposite said vessel portion.

6. An implant according to claim 5 further comprising a load distributing member positioned between said sheet of flexible material and said epicardial surface.

7. An implant according to claim 4 wherein said anchor material is a plurality of strips of flexible material extending radially from said conduit and adapted to extend over an epicardial surface of the myocardium wall at a region positioned opposite from the vessel portion.

8. An implant according to claim 7 further comprising a load-distributing member positioned between said sheet of flexible material and said epicardial surface.

9. An implant according to claim 1 wherein said anchor includes a rigid member secured to said conduit and extending therefrom opposite said vessel portion for said rigid member to overlie an epicardial surface of the myocardium wall at a location opposite said vessel portion.

10. An implant according to claim 9 wherein said rigid member is at least partially covered with a tissue growth inducing material.

11. An implant according to claim 1 wherein said anchor extends in a direction the same as said vessel portion and includes attachment locations positioned on opposite sides of said vessel portion for attachment to an epicardial surface of the myocardium wall on opposite sides of said coronary vessel.

12. An implant according to claim 11 wherein said anchor includes a rigid brace secured to said conduit and having first and second arms extending therefrom on opposite sides of said vessel portion and terminating at said attachment locations.

13. An implant according to claim 12 wherein said brace is selectively positionable on said conduit.

14. An implant according to claim 1, wherein the anchor includes at least a portion that extends transversely outwardly from the myocardial portion.

15. An implant according to claim 1, wherein the anchor includes at least a portion that extends outwardly from the myocardial portion in a direction opposite from the vessel portion.

16. An implant according to claim 1, wherein the vessel portion is sized to be received within the vessel.

17. An implant according to claim 1, wherein the anchor includes a sheet of flexible material connected to the conduit at a location adjacent the vessel portion.

18. An implant according to claim 1, wherein the anchor includes a rigid arm connected to the conduit at a location adjacent the vessel portion.
